(19) United States
(12) Patent Application Publication
(10) Pub. No.: US 2011/0076307 A1
(43) Pub. Date: Mar. 31, 2011

(54) POLYCATIONIC GENE CARRIERS FORMED OF ENDOGENOUS AMINO GROUP-BEARING MONOMERS

(76) Inventors: Tuo Jin, Highland Park, NJ (US); Zixiu Du, Shanghai (CN)

(21) Appl. No.: 12/843,691

(22) Filed: Jul. 26, 2010

Related U.S. Application Data
(63) Continuation-in-part of application No. PCT/CN2009/000116, filed on Jan. 24, 2009.
(60) Provisional application No. 61/087,958, filed on Aug. 11, 2008.
(60) Provisional application No. 61/023,426, filed on Jan. 25, 2008.

(51) Int. Cl. A61K 9/00; A61K 31/7105; A61K 31/711; A61P 43/00; C08G 63/685; C08G 69/00; C08G 63/91; C08G 73/06; C08G 73/02

(52) U.S. Cl. 424/400; 514/44 R; 528/363; 525/418; 528/327; 528/245

(57) ABSTRACT

The present invention is directed to a design of and a method to synthesize polycations for gene (DNA and RNA) delivery. According to this design, the polycations (also said cationic polymers) are formed by polymerization of endogenous monomers bearing sufficient amino groups through degradable bonds with linker molecules or with themselves. The amino group-bearing monomers are those naturally existing in or nontoxic to human body. The linker molecules are those which are not only degradable to nontoxic fragments but also able to release the amino group-bearing monomers in their native state upon degradation. Some examples for the endogenous amino group-bearing monomers are spermine, spermidine, serine or N,N-dimethyl serine, and histidine. Examples for the degradable chemical bonds formed between the amino group-bearing monomers are carbamate, imine, amide, carbonate, and ester. In order to improve degradability or proton sponging effect, low pKa (<8) amino group(s) or other electron donating group(s) is incorporated in the linker between the two (or three) reactive groups for linking the amino group-bearing monomers. These polycationic carrier systems can be used for nano-encapsulation and transfection of gene materials.

### CROSS REFERENCES TO RELATED APPLICATIONS

This application is a Continuation-In-Part Application of International Application No. PCT/CN2009/000116, filed Jan. 24, 2009, which claims benefit of U.S. Provisional Application No. 61/087,958 filed Aug. 11, 2008 and U.S. Provisional Application No. 61/023,426, filed Jan. 25, 2008. The entire contents and disclosures of the preceding applications are incorporated by reference into this application.

Throughout this application, reference is made to various publications. The disclosures of these publications, in their entireties, are hereby incorporated by reference into this application to more fully describe the state of the art to which this invention pertains.

### FIELD OF THE INVENTION

This invention demonstrates a design and a method to synthesize and assemble polycationic gene (and RNA) carriers degradable to endogenous monomers.

### BACKGROUND OF THE INVENTION

It has been sufficiently evident that polynucleotides of sensible sequences can be used as effective therapeutic agents in drug therapy, vaccination and tissue regeneration by turning the relevant gene on (expression) or turning it off (silencing)^{[1]}. To achieve these therapeutic efficacy, however, therapeutic genes, DNA vaccines as well as siRNA drugs must be delivered to the nuclei or cytoplasm of target cells. Among the carrier systems for delivering polynucleotides (gene materials), synthetic delivery systems possess a series of advantages over viral vectors such as freedom from immunity and viral mutation, ability to package multiple genes or siRNA of choice into particulate vehicles via a single mechanism, and adaptability to simple and cost-efficient manufacturing process^{[2]}. To deliver gene materials (DNA and RNA) to targeted inter- and intra-cellular sites effectively using non-viral systems, the synthetic gene carriers (i.e. non-viral vectors) must play a number of virus-like functions including packing and condensing gene materials, cell targeting and entering, endosomal escaping, and release of genes in cytoplasm. Finally, the carrier system itself must be metabolized to small, non-toxic species that can be eliminated from the body. If any of the above functions is lacking, the corresponding step will become the rate-limiting barrier of the entire mechanism of gene transfection. In addition, the synthetic gene carrier itself must be nontoxic, biocompatible and able to be metabolized. However, none of the synthetic gene delivery systems reported to date meets all these criteria.

Synthetic gene delivery vehicles reported in last decades can, in general, be divided into several categories, cationic liposome-based systems (called lipoplex), cationic polymer-based systems (called polyplex), lipid-cationic polymer combined systems (called lipopolyplex) and non-charged nanometer particulates. The majority of them are lipoplexes and polyplexes due to the negative charges of DNA and RNA by which the gene materials may easily be condensed with particles with positively charged liposomes or polymers. These two categories possess different advantages and mechanisms in terms of each step of gene transfection. Cationic liposomes condense gene materials less compactly than cationic polymers^{[3]} but offer unique membrane fusion function with endosomes that may help DNA or RNA to escape to cytoplasm in molecular form^{[4]}. Polycations (cationic polymer), on the other hand, may condense gene materials in more compacted form^{[3]} so that better protection and larger capacity of gene materials are expected^{[5]}. For endosomal escaping, polyplex is believed to undergo a “proton sponging” process for which the polyplex-engulfing endosome is ruptured by chloride ions accumulated due to continuous influx pumping of HCl to compensate for protons consumed by the cationic polymer carrier. In this case, the protonated polycation may hold DNA or RNA even tighter within polyplex (due to increased positive charges) so that the gene materials enter cytoplasm in the form of particles rather than molecules. However, the polynucleotides must be released or extracted out of the polyplex in order to exert their biological functions. It seems that condensation and release of DNA or RNA by polycations are a pair of contradictory processes which require a polycationic carrier system to be chemically dynamic and biologically responsive.

To compromise gene packing capacity and cytoplasm release, some researchers suggested to use or design a polycationic carrier which possesses a mild strength of gene condensation^{[6]}. Using a cationic polymer with low molecular weight or with low amino group density is one of the approaches^{[7]}. Another strategy is to use environment responsive polycations to achieve gene condensation and releasing, the two opposite moves^{[8]}. This type of polymers are, however, often complex in structures and complicated in metabolic process and metabolized products. Using degradable cationic polymers as gene carriers may be a more reasonable approach by which gene release may be achieved by degradation of the backbone of the carriers, a process independent of its ability to condense DNA or RNA^{[9]}. In addition, degradation to small molecules will reduce chemical toxicity of polycations. As reported in the literature, biodegradable linkages such as carboxylic ester, phosphate ester, imine or disulfide structure were incorporated in the backbone of a cationic polymers. In this aspect, ester bond is the most widely used degradable structure to incorporate into the polycation backbone for its balanced stability and degradability. However, ester bond is highly reactive to nucleophiles such as primary and secondary amino groups^{[10]}, which are the key functional groups for gene compacting and proton sponging.

To avoid the ester-amino group reaction, two strategies were used in previous studies, synthesizing cationic polymers with only tertiary amino groups or using di-sulfide structure to form the degradable polymer backbone^{[11-13]}. For example, some researchers polymerized branched small molecular polyethylenimine (PEI) via an ester-bearing linker, and that the cross-linked small molecular PEI carriers possess higher gene transfection efficiency but lower toxicity^{[13]}. Backbone degradation of this polymer was achieved by cleavage of the linker, leaving the cleft fragments attached to the small molecular PEI or other amino group-bearing monomers (the polymer building blocks)^{[11,12]}. Such a backbone degradation pattern may be fine for a polycationic gene carrier formed of man-made amino group-bearing building blocks. For a degradable cationic polymer formed of endogenous amino group-bearing monomers, the attachment of linker fragments upon polymer degradation will dismiss the advantages of using endogenous monomers. A polycationic gene carrier degradable to human endogenous amino group-bearing monomers is an ideal design to achieve intracellular release of genes and metabolic elimination of the carrier itself.

The primary objective of this invention is to develop polycationic gene carriers which possess sufficient amount of amino groups to condense polynucleotides into compacted particles and to induce endosomal break through proton sponge effect^{[13]}, and possess fully degradable backbone to release polynucleotides after endosomal escape and to turn itself to endogenous or non-toxic metabolites.

### SUMMARY OF THE INVENTION

As discussed above, a clinically useful delivery system should be capable of packing DNA or RNA of choice (single or multiple types) into nanoparticles with sufficient density, to target the polynucleotide-loading nanoparticles to diseased cells, to transport and release gene materials into cytoplasm of the cells, and finally, to degrade itself to nontoxic metabolites. For practical applications, the system should best be simple in structure, easy to prepare and formulate, and stable in storage, transportation and clinical operation. The above biological criteria may be translated into a number of chemical properties of a synthetic polycationic carrier, including sufficient positive charge to pack negatively charged DNA or RNA, flexibility and easiness to conjugate targeting moieties for diseased cells, carrying sufficient amount of low pKa (<8) amino groups as a pool for proton sponging, and degradability to non-toxic (preferably endogenous) monomers for intracellular release of polynucleotides and metabolic elimination of the carrier self.

The present invention discloses a design of chemical structures of cationic polymer of which endogenous monomers bearing sufficient number of amino groups are polymerized by forming degradable bonds with linker molecules or with themselves. The amino group-bearing monomers are those naturally existing in or nontoxic to the human body. The linker molecules are those which are not only degradable to nontoxic species but also able to release the amino group-bearing monomers in their native state upon degradation. Some examples for the endogenous amino group-bearing monomers are spermine, spermidine, serine or N,N-dimethyl serine, histidine and arginine Examples for the degradable chemical bonds formed between the amino group-bearing monomers and the linker molecules are, but not limited to, carbamate, imine, amide, carbonate, and ester. In order to improve degradability or proton sponging effect, low pKa (<8) amino group(s) or other electron donating group(s) is incorporated into the linker molecule between the two (or three) reactive groups for linking the amino group-bearing monomers.

### BRIEF DESCRIPTION OF FIGURES

FIG. 1. Cationic polymer (Polylink-SP) polymerized through carbamate linkages between amino group-bearing monomers and linker molecules. A: Examples of the repeating units of Spermine-based polymers; B: Examples of the repeating units of Spermidine-based polymers. The linker molecule may form carbamate linkage with any amino group of spermine or spermidine, and the ends of the polymer chain may be an amino group-bearing monomer or a linker molecule, or capped by any other chemical structures capable of reacting with said monomer or linker molecule.

FIG. 2. Polycations (PolyLink-SP) polymerized through carbamate linkages between the amino group-bearing monomers and the linker molecules. The carbamate bonds are formed between the linker molecule and the secondary amino group of spermine (for which the two primary amino groups were protected prior to polymerization and deprotected afterwards). A: Polyspermine carbamate formed by condensation of primary-amino-protected spermine and 1,4-butanediol bischloroformate, followed by deprotection; B: Polyspermine carbamate formed by condensation of primary-amino-protected spermine and ethylene bischloroformate, followed by deprotection. The open end of the polymer chain in A and B may be an amino group-bearing monomer or a linker molecule, or capped by any other chemical structures capable of reacting with said monomer or linker molecule.

FIG. 3. Polyspermine amide (Polyamide-SP) formed by condensation between spermine and succinic chloride. The linker molecule may form carbamate linkage with any amino group of spermine or spermidine, and the ends of the polymer chain may be an amino group-bearing monomer or a linker molecule, or capped by any other chemical structures capable of reacting with said monomer or linker molecule.

FIG. 4. Polyspermine carbamate formed by condensation of spermine and ethylene bischloroformate. A: one end of the polymer is conjugated with cholesterol (Cho-Polylink-SP), the other end may be conjugated with the same or any other chemical structures capable of reacting with spermine or the linker molecule; B: one end of the polymer is conjugated with PEG (PEG-Polylink-SP), the other end may be conjugated with the same or any other chemical structures capable of reacting with spermine or the linker molecule.

FIG. 5. Polyspermine amide (Polyhistidine-SP) formed by condensation of spermine and activated histidine succinate linker. The linker molecule may form carbamate linkage with any amino group of spermine or spermidine, and the ends of the polymer chain may be an amino group-bearing monomer or a linker molecule, or capped by any other chemical structures capable of reacting with said monomer or linker molecule.

FIG. 6. Polyspermine imine (Polyimine-SP) formed by condensation between the primary amino groups of spermine and bisaldehyde linker to form an imine linkage. A: Polyspermine imine polymerized with glyoxal linker; B: Polyspermine imine polymerized with glutaraldehyde. The ends of the polymer chain may be an amino group-bearing monomer or a linker molecule, or capped by any other chemical structures capable of reacting with said monomer or linker molecule.

FIG. 7. Synthetic scheme of poly-pseudo-serine, wherein R₁ and R₂ are methyl groups, and R′ is PEG, target moiety or hydrophobic anchor.

FIG. 8. Polyspermine carbamate polymerized by condensation between spermine and a bischloroformate linker with an imidazole group incorporated in the linker between the two carbamate bonds.

FIG. 9. Molecular weight and morphological changes of Polylink-SP incubated in HEPS buffer (pH=7) at 37° C. for various days. A: GPC-HPLC charts describing molecular weight of Polylink-SP incubated for 0, 5 and 7 days; B: Morphology of Polylink-SP samples after incubation and lyophilization.

FIG. 10. Electrophoresis and Zeta potential measurement of polyplex as a function of polymer/DNA ratio. A) Electrophoresis of GFP DNA mixed with Polylink-SP; B) Zetapotential of Polyplex formed of Polylink-SP and GFP DNA.

FIG. 11. Particle sizes of polyplex formed of Polylink-SP and GFP plasmid. A: in saline; B: in pure water.

FIG. 12. Transfection activity of Luciferase genes. A. Polyspermine carbamate; B: PEG-polyspermine carbamate.

FIG. 13. Viability of COS-7 cells treated with Polylink-SP, PEGylated Polylink-SP and PEI-25 KDa

FIG. 14. Microscopic images of COS-7 cells: Green fluorescent dots: polyplexes with fluorescent labeling; dark blue patches: nuclei of COS-7 cells.

FIG. 15. Assembly of lipid bilayer around polyplex formed with spermine-based or serine-based cationic polymers conjugated with hydrophobic anchors. The hydrophobic anchor molecules may be any hydrophobic molecule with molecular weight over 200, such as fatty acids and cholesterol.

FIG. 16. Activity of various nucleotide carrier systems in delivering EGFP silencing siRNA. The EGFP expression amounts are normalized to that from the untreated cells.

### DETAILED DESCRIPTION OF THE INVENTION

Effective gene delivery requires a delivery system to accomplish a series of biological functions including condensing genes into compacted particles, carrying genes into target cells, helping genes to escape endosomal degradation, releasing genes into cytoplasm, and degrading the delivery system into monomers non-toxic to and able to be eliminated from the body. To meet these requirements, a synthetic gene delivery system should, structurally, include respective functional groups to exert the above-mentioned biological functions. To simplify preparation process and toxicity check-out, it is also important that a synthetic gene carrier is structurally simple for which each functional group of choice should address multiple biological tasks. For example, it is desirable to possess a sufficient number of amino groups of desired pKa in the carrier to enable the genes to condense into nano-particles, to exert proton sponging effect for endosomal escape, and not to generate too much surface charges in the polyplex for better circulation and cell-targeting. The backbone of the carrier should degrade at appropriate rate during gene transfection to release compacted genes in cytoplasm, to minimize cytotoxicity resulted from polycations, and to generate free amino groups to facilitate endosomal escape. The carrier should also have a functional group to which various functional groups such as targeting moieties, circulation improvers (PEG for example), and lipid membranes can easily be attached. The present invention is aimed at creating such a delivery system by bio-function-based chemical design.

One embodiment of the gene carrier system of the present invention consists of two cationic polymers formed by endogenous amino group-bearing monomers, spermine and N,N-dimethyl serine. One of the polymer is formed by linking spermine with a linker molecule through a degradable bond between spermine and the linker. The degradable bonds can be carbamate, amide or imine. For polycarbamate or polyamide, the two primary amino groups of spermine may be protected prior to reaction with bischloroformate or bisacid chloride, respectively, to achieve better defined linear polymer. For polyimine, the imine bond is formed selectively between the primary amines of spermine and the aldehyde groups of the linker.

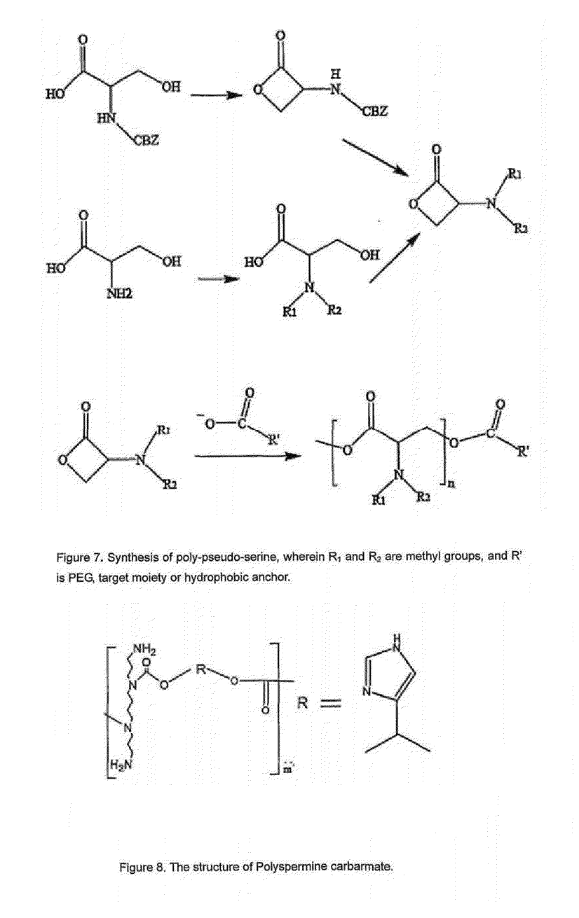

One problem for polyspermine carbamate and polyspermine amide is that degradation rate of the carbamate linkage and the amide linkage is too low to release polynucleotides and molecular spermine in cells. Prolonged exposure of the body to cationic polymers is believed to be a source of toxicity. To facilitate degradation of polyspermine carbamate and polyspermine amide, an electron donating group (such as imidazole or histidine) may be incorporated in the linker molecules. In addition to better degradability, polyplexes formed by gene materials and polyspermine carbamate or polyspermine amide involving low pKa imidazole or histidine possess less positive charges for the total number of amino groups required for proton sponging. The molecular structures of histidine-incorporated polyspermine amide and imidazole-incorporated polyspermine carbamate are shown in FIGS. 5 and 8, respectively.

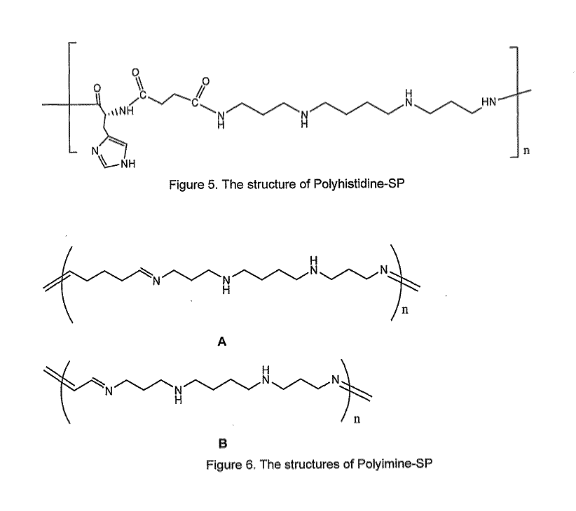

The problem for polyspermine imine is, on the contrary, the poor stability of the polyimine linkage. To improve stability of polyspermine imine, bisethylene aldehyde was used as the linker to polymerize spermine. The molecular structure of polyspermine ethyleneimine is shown in FIG. 6. One hypothesis is that the two C═N double bonds form a conjugated π structure, thus stabilizing the imine linkage.

As reaction pathway, spermine polymerization through the chemical linkage is achieved by dropping bischloroformate, bisbromoformate, metaformaldehyde or glyoxal into a solution of the amino group-bearing species stepwise. The molecular weight of the polymers can be controlled by the molar ratio of the amino group-bearing species over the linker molecules, and by selection of solvent for the reaction.

Another cationic polymer used in assembling polyplexes is poly-pseudo-N,N-dimethyl serine. The molecular structure and synthetic pathway of this cationic polymer are shown in FIG. 7. A unique feature of poly-pseudo-N,N-dimethyl serine, in addition to better degradability and reasonable stability, is its synthetic pathway. N,N-dimethyl serine is first dehydrated to form a lactone through Mitsunobnu reaction. Then the 4-membered ring lactone is allowed to react with a nucleophilic polymerization initiator to polymerize via continuous ring opening. Since the 4-membered ring lactone is highly reactive to anionic ring opening polymerization (to form ester bond) and the initiator can be a carboxylic ion bonded to almost any chemical group, functional groups (fatty acids, cholesterol, PEG or targeting moieties) attached to the carboxylic group can easily be conjugated to one end of the poly-pseudo-N,N-dimethyl serine as the polymerization initiator. End group attachment of a functional group to a cationic polymer is favorable in terms of exposing this group at the surface when forming polyplex with DNA or RNA. The attached fatty acids or cholesterol is for forming polyplex with hydrophobic surface anchors. It was reported that hydrophobic surface anchors induced lipid bilayer formation around particles [14]. The hydrophobic groups conjugated on the polyplex surface induce self-assembly of a lipid bilayer around the particle to form lipopolyplex more stable than previously reported lipopolyplexes formed by ionic adsorption (Refer to FIG. 15).

Poly-pseudo N,N-dimethyl serine possesses a polyester backbone and thus has balanced degradability/stability as compared with polyspermine carbamate, polyspermine amide and polyspermine imine. The pKa of its amino group should be substantially lower than 9.15, the pKa of the amino group of serine due to esterification of serine's carboxylic group. The amino groups of poly-pseudo N,N-dimethyl serine are all tertiary amines which are reported by some researchers to be favored for proton sponging. One drawback for poly-pseudo N,N-dimethyl serine is that its degradation generates carboxylic groups instead of amino groups upon backbone cleavage. Acid generation normally compromises proton sponging effect. Therefore, preparing polyplex or lipopolyplex using both the serine-based and the spermine-based polycations as combined gene packing system may be a better choice.

Gene materials (DNA or RNA) can be condensed into particles simply by mixing a solution of genes with an aqueous solution of the polycations at appropriate nitrogen to phosphor ratio. Gene transfection, anti-sense effect or RNA interferon effect can be achieved by adding this gene carrier suspension into cell medium. The polycations help polynucleotides to enter cells, escape from endosomes, and get released into cytoplasm.

Functional groups (such as targeting moieties) can be conjugated directly to the polycations synthesized above, or to another polycations made of amino acids (poly-pseudo serine derivatives). Unlike most polycations that are synthesized by condensation, poly-pseudo serine derivative is synthesized through anionic ring opening. The functional groups to be conjugated can function as a polymerization initiator to which the monomers of serine lactone is added one by one through ring opening reaction. By this mechanism, the functional group is conjugated at the end of the polymer chain, easily to be exposed at the surface of gene-polycation polyplex particles (FIGS. 5 and 7).

The polycations possess great polynucleotide condensation capacity as they can simply be mixed with a large or small gene, single type or multi-type of genes, to form nanometer particles for cell to take in. The linked small molecular PEI or linked spermine may function as gene condenser and proton sponge, while the poly-pseudo serine derivative may function as polyplex surface spacer to conjugate targeting moieties.

Another advantage of the linked small molecular PEI and linked spermine (in addition to lowering toxicity) is that their degradation does not generate acidic groups like other degradable polymers. Rather, their degradation generates free amino groups that help to buffer the acidity inside endosomes. This property helps to break endosome and release polynucleotides into cytoplasm without initial amino group density (which leads to lower surface charge of polyplex). For proton sponge effect, the endosome is broken by osmotic pressure generated by proton sponging effect (absorption of protons). Amino groups of the polycations are responsible for proton absorption. However, increase in amino group in cationic polymer (i.e. N/P ratio) will also lead to positive surface charges of polyplex which reduces circulation time of the polynucleotide-carrying particles in the body due to the negative charge of tissue surfaces. In the present invention, some amino groups form degradable carbamate, urea or imine structure. These nitrogen-containing linkages do not contribute to positive surface charge when it condenses with gene materials, but offer acid buffering effect when they degrade in endosomes and release free amino groups after being taken by cells. This nature helps the polynucleotide-carrying particles to achieve the same endosomal escape effect with lower surface charge, i.e. better targeting ability.

The present invention provides a cationic polymer comprising a plurality of amine monomers linked together by themselves via cleavable bonds, or by way of linkers, wherein each linker forms cleavable bonds with two or three said amine monomers. The amine monomers may be human endogenous amines or their derivatives. In one embodiment of the invention, the amine monomers are selected from spermine, spermidine, serine, N,N-dimethylserine, amino acids, alkylated or dialkylated amino acids, histidine and their combinations thereof. In one embodiment of the invention, the cleavable bonds can be cleaved to release said amine monomers. In another embodiment of the invention, the cleavable bonds are esters, amides, carbamates or imines. In the above-mentioned cationic polymer, the linkers may comprise an amino group with pKa<8. In one embodiment, the amino group with pKa<8 is an imidazole, amino acid or histidine.

In one embodiment of the invention, the cationic polymer is conjugated with one or more biologically functional groups. The one or more biologically functional groups may be selected from fatty acids, cholesterol succinate, phospholipids, polyethylene glycol (PEG) and cell-targeting moieties.

In one embodiment of the invention, the cleavable bonds of the cationic polymer are formed by reaction between the amine monomers with the following moieties in the linkers: carboxylic acid, aldehyde, chloroformate, acyl halide, activated ester, or a combination thereof.

The present invention also provides a method of synthesizing the above-mentioned cationic polymer, comprising a reaction between the amine monomers and the linkers possessing two or three reactive groups, or a reaction between said amine monomers. Said reactive groups may be chloroformates, aldehydes, carboxylic acid halides or activated esters.

In one embodiment of the invention, the amine monomers react with themselves through ring opening polymerization or through activated acid-hydroxyl condensation.

The present invention further provides a use of the cationic polymer to encapsulate and deliver a DNA or RNA. In one embodiment of the invention, the cationic polymer may be used to assemble lipid bilayers around polyplex through hydrophobic interactions.

The following examples are for illustrative purposes only and should not be construed to limit the scope of the present invention. It is well known in the art that a polymer may be capped with a diverse array of molecules that can react with the remaining reactive groups on the polymer. This capping step is omitted in some of the examples below.

### EXAMPLES

### Example 1

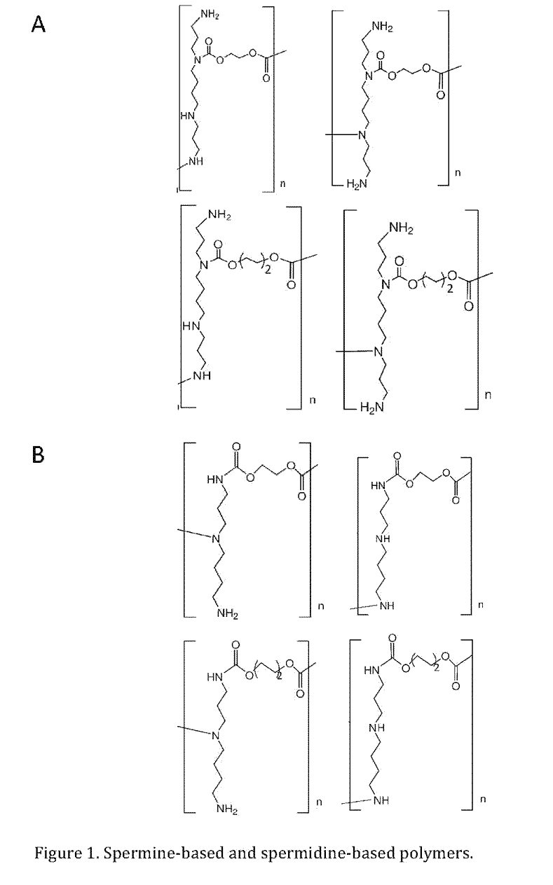

### Synthesis of Polyspermine Carbamate (See FIGS. 1a and 1b)

To polymerize spermine via carbamate linkage, 1 equivalent of ethylene bischloroformate or 1,4-butanyl bischloroformate dissolved in chloroform was added dropwise to a stirring solution of spermine dissolved in chloroform and triethylamine at 0° C. under a nitrogen stream. The solution was then warmed up to room temperature and stirred for 12 h. After removal of the solvent by evaporation, the obtained polymer pellet was dissolved in water and dialyzed to remove fragments with molecular weight of less than 3500. The final product was stored at −20° C. after lyophilization.

### Example 2

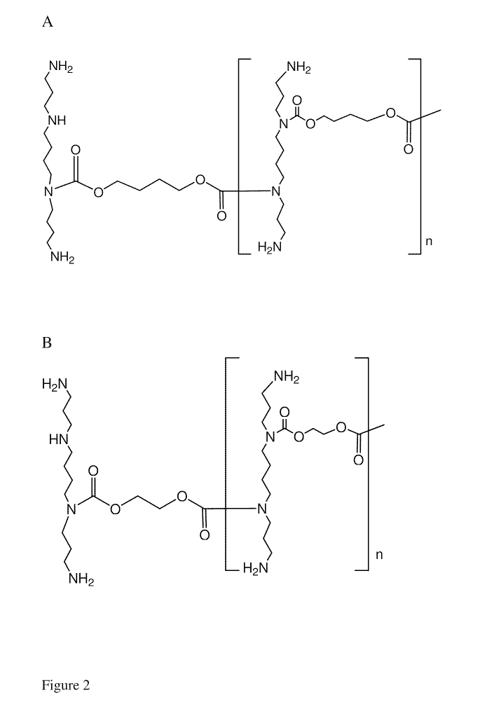

### Synthesis of Linear Polyspermine Carbamate (See FIGS. 2a and 2b)

To synthesize linear polyspermine carbamate, the two primary amino groups of the reactant was protected by adding ethyl trifluoracetate to a spermine solution (in methanol) dropwise at −78° C. under a nitrogen stream, followed by continuous stifling at 0° C. for 1 h. The product, N¹,N¹⁴-bis(trifluoroacetyl)spermine, was obtained after evaporating the solvents, and subjected to the same polymerization steps described in Example 1. After polymerization, the trifluoroacetate group was removed by treating the polymer (dissolved in methanol) with 30 wt % aqueous NH₃ solution (in a sealed container) at 60° C. for 8 h. The obtained cationic polymer was then dialyzed to remove small molecular fragments with molecular weight less than 3500.

### Example 3

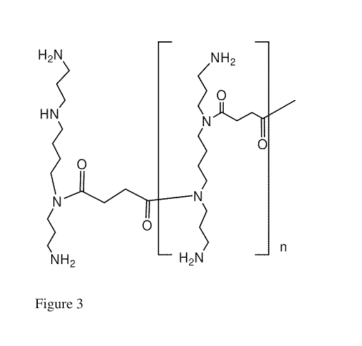

### Synthesis of Linear Polyspermine Amide (See FIG. 3)

The reaction procedure for synthesis of linear polyspermine amide was the same as that of polyspermine carbamate, except that 1 equivalent of ethylene bischloroformate or 1,4-butanyl bischloroformate was replaced by 1 equivalent of succinyl chloride.

### Example 4

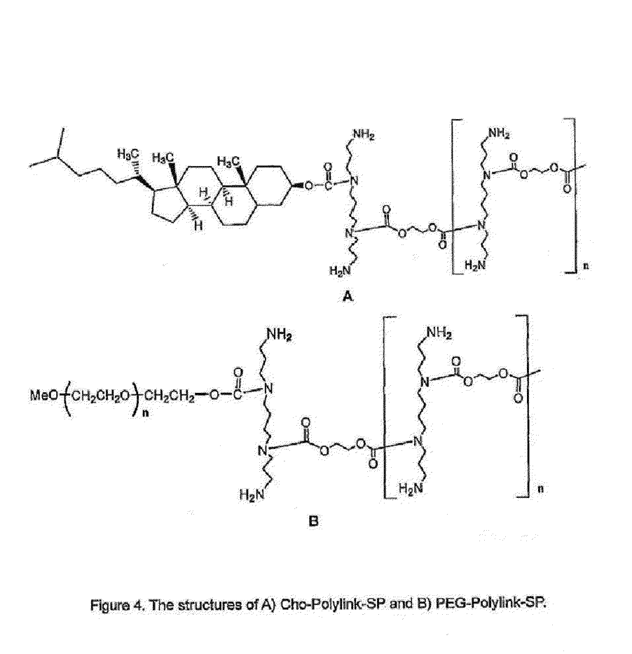

### Synthesis of Cholesterol- or Peg-Conjugated Linear Polyspermine Carbamate (See FIG. 4)

To conjugate cholesterol or PEG to polyspermine carbamate, the polymer synthesized as in Example 2 was treated prior to deprotection by adding mPEG-SC (5000) or cholesteryl chloroformate solution (dissolved in dry chloroform) dropwise into the polymer solution. Since only the two ends of the polymer chain possess unprotected amino groups, mPEG or cholesterol groups are expected to conjugate at the chain ends. The later steps, deprotection, lyophilization, re-dissolving and dialysis, were the same as Example 2.

### Example 5

### Synthesis of Polyspermine Histidine (See FIG. 5)

To synthesize the intermediate imidazole-containing linker, succinic anhydride was added to histidine dissolved in sodium alcoholate at 60° C., followed by refluxing for 6 h. The temperature was then reduced to 50° C. and hydrochloric acid was added to terminate the reaction. The product was purified by recrystallization from acetone. The imidazole amino was protected with BOC. To synthesize Polyspermine histidine, the spermine was added to the intermediate dissolved in buffer solution at 50° C. and stirred for 2 h for polymerization. The BOC was removed after polymerization and the obtained cationic polymer was then dialyzed through a membrane of desired cut-off size to remove small molecular fragments.

### Example 6

### Synthesis of Polyspermine Imine (See FIG. 6)

Glyoxal (˜40 wt % in the aqueous solution) or glutaraldehyde (˜45 wt % in the aqueous solution) solution was added to spermine dissolved in anhydrous ethanol dropwise at 0° C. under a nitrogen atmosphere. The reactants were then stirred at room temperature overnight. The reaction system was evaporated to remove the solvent and the obtained cationic polymer was dialyzed through a membrane of desired cut-off size to remove small molecular fragments.

### Example 7

### Degradability of Polyspermine Carbamate

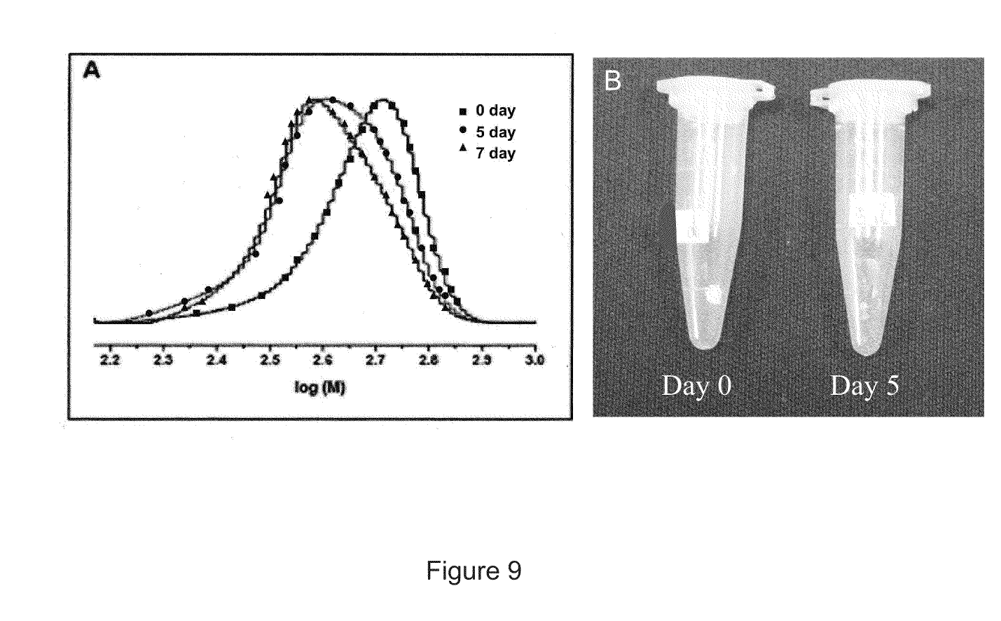

Degradability of Polylink-SP (polyspermine carbamate) was examined by incubating the polymer in HEBS buffer (pH=7) at 37° C., followed by molecular weight analysis using GPC-HPLC at determined days of degradation incubation. To double confirm degradation of Polylink-SP, the samples undergoing various days of degradation were lyophilized for morphological observation. Results of the two experiments are shown in FIGS. 9A and 9B, respectively.

### Example 8

### Formation and Physical Chemical Characterization of Polyplex Formed from Polyspermine Carbamate and DNA

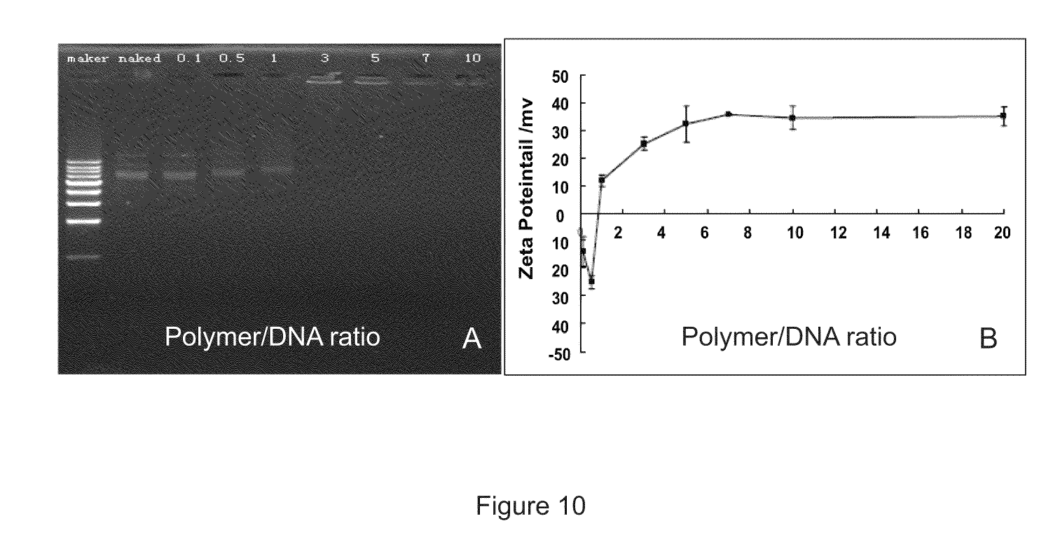

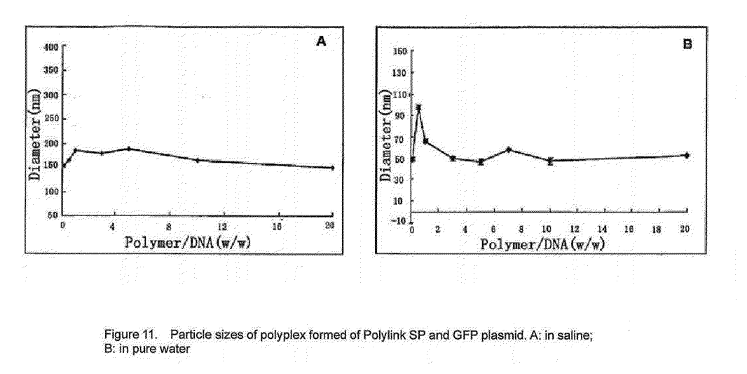

Capability of the designed cationic polymer (Polylink-SP) to condense polynucleotides to polyplex was examined as a function of weight ratio of polymer/DNA using electrophoresis. A stock solution of Polylink-SP was added to a solution of green fluorescent protein plasmid at different polymer to DNA ratio. The samples were loaded on an electrophoresis plate for analysis. The same samples were also subjected to Zeta potential measurement, and the results of the two measurements are shown in FIGS. 10A and 10B respectively.

### Example 9

### Particle Size of Polyplex Formed of Polylink SP and DNA

The hydrodynamic sizes of the polyplex formed as above were determined by dynamic light scattering at 25° C. using a 4.0-mW He—Ne laser (λ=633 nm) as the incident beam at a scattering angle of 90°. The results of the two measurements are shown in FIGS. 11A and 11B respectively.

### Example 10

### Transfection Activity of Report Genes Delivered by Polylink-SP

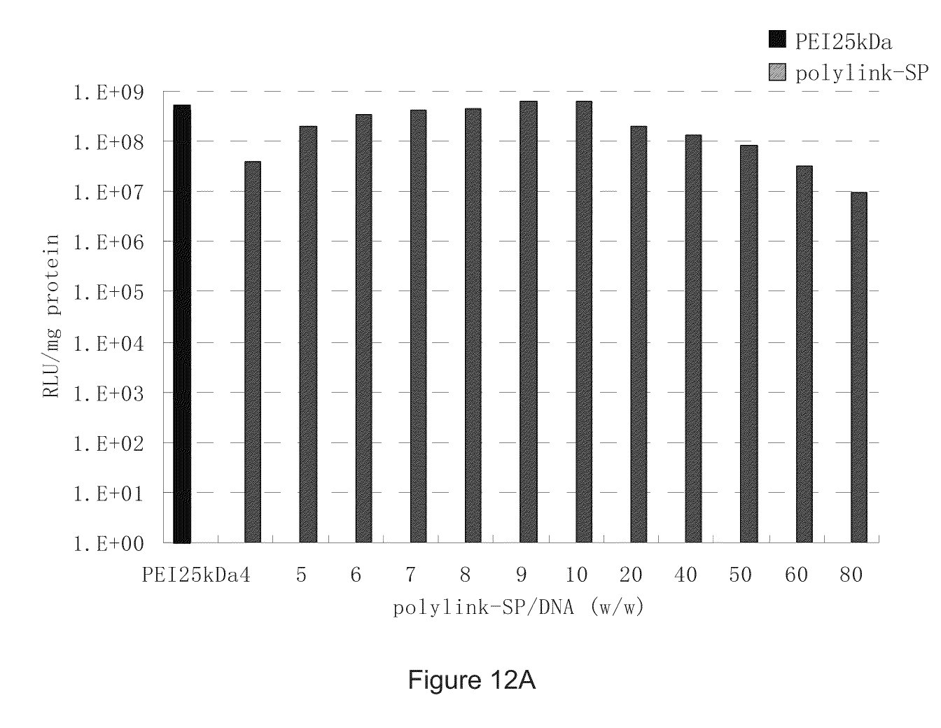

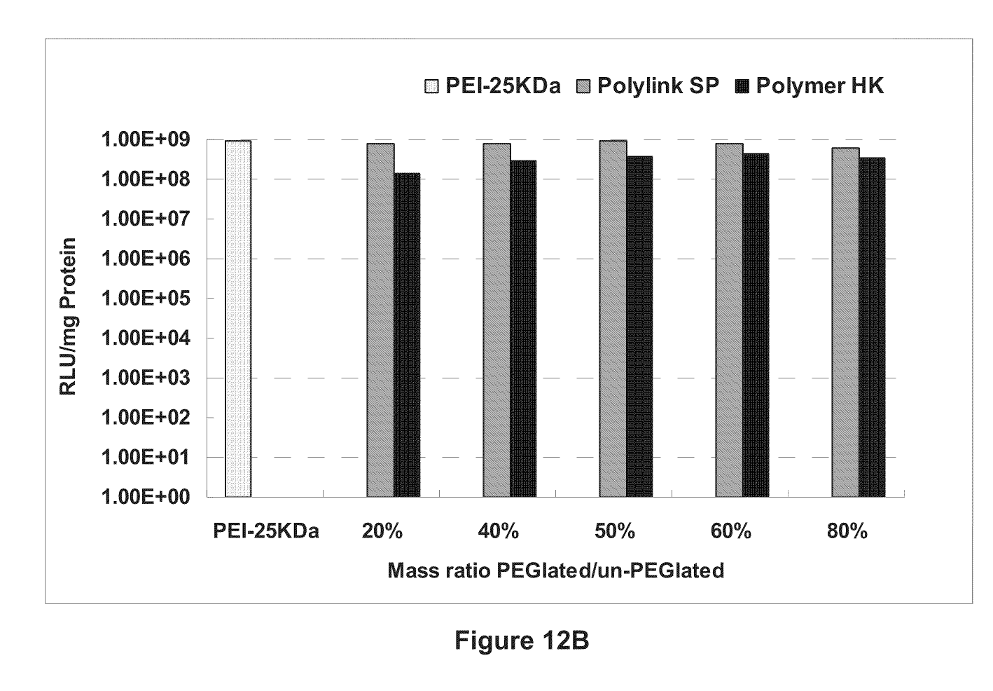

The activity of Polylink-SP in transfection of luciferase gene (one most frequently used for report genes) in COS-7 cells was compared with PEI-25 KDa, one of the most frequently used reference. At optimized polymer to gene ratio (7-10), Polylink-SP showed a transfection activity comparable to that of PEI-25 KDa (FIG. 12A), suggesting considerable transfection efficiency of the synthesized polymer.

Transfection efficiency of Polylink-SP was also compared with another reference, HK polymer, a polypeptide formed by co-polymerization of lysine and histidine, in luciferase gene expression (FIG. 12B). The polymer to gene ratio was 10 for Polylink-SP and 12 for HK polymer. In order to determine the effect of PEGylation on gene transfection activity, PEGylated Polylink-SP (containing 36% PEG in weight fraction) was mixed into both Polylink-SP and HK polymer (from 20% to 80% by weight). At low fraction of PEGylated Polylink-SP (20% by weight), Polylink-SP showed a transfection activity similar to that of PEI-25 KDa but an order of magnitude higher than that of HK polymer. As the fraction of PEGylated Polylink-SP increase (from 20% to 80% by weight), the activity of Polylink-SP decreased slightly, while that of HK polymer increased gradually, reflecting that the two mixture approached to pure PEGylated Polylink-SP from two sides (FIG. 12B). For Polylink-SP, mixing 50% by weight of PEGylated polymer into it did not affect transfection efficiency for luciferase genes by 18% of PEG in mass fraction (FIG. 12B). In fact, the luciferase gene transfection activity of the PEGylated Polylink-SP was comparable to that of un-PEGylated sample (FIG. 12B).

### Example 11

### Cytotoxicity of Polyspermine Carbamate and PEGylated Polyspermine Carbamate

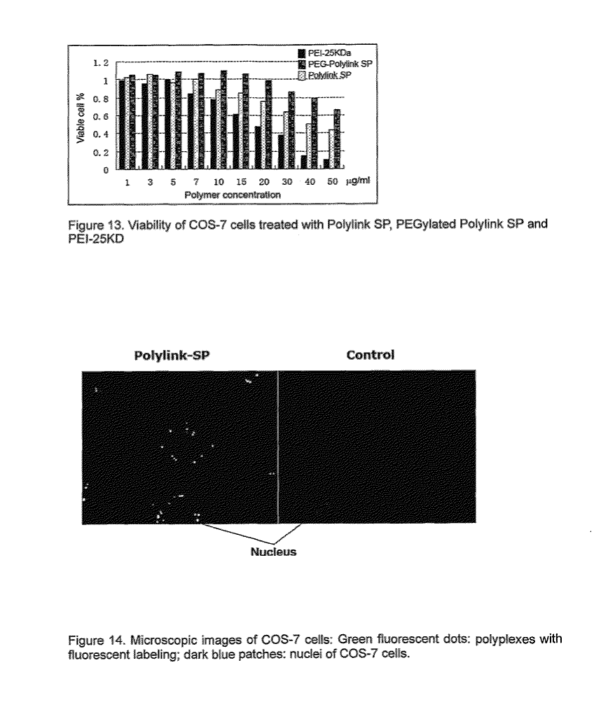

Cytotoxicity of the polymer was examined using a MTT assay in comparison with the 25 kDa PEI. The COS-7 cells were seeded in a 96-well plate at a density of 10⁴ cells per well in 100 μl of growth medium for 24 h. Then the growth medium was replaced with fresh, serum-free and phenol red-free medium containing the polymer. Cells were incubated with polymer for 4 h, and the medium was replaced with complete DMEM and 25 μl of 5 mg/ml MTT solution in PBS buffer. The results of the two measurements are shown in FIG. 13.

### Example 12

### Location of Polyplex Formed of Polyspermine Carbamate and Fluorescent siRNA

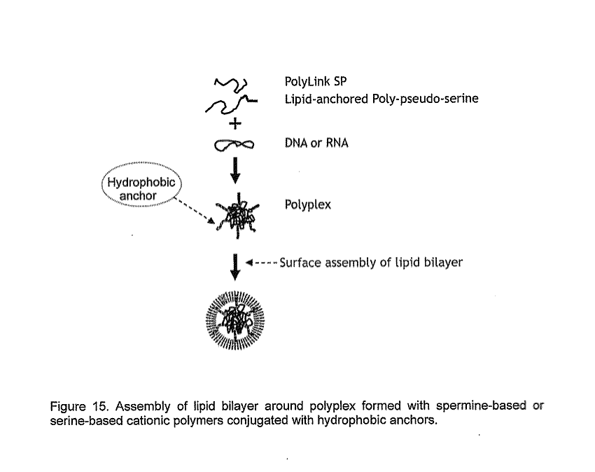

To clarify whether Polylink-SP (polyspermine carbamate) may carry siRNA out of endosomes, fluorescent labeled siRNA was used to form polyplex with the polymer and incubated with COS-7 cells. The cell nuclei were dyed and the transfected cell culture was examined under a confocal microscope. As shown in FIG. 14, fluorescent polyplex was adsorbed around the nucleus of the cells, suggesting that the Polylink-SP can effectively carry siRNA into cells and out of endosomes. The fluorescent labeled polyplex above was also added to a culture of lever cells which were found to show similar fluorescent dots, indicating that the polyplex particles had entered the cells.

### Example 13

### Formation of Lipopolyplexes Via Hydrophobic Interaction

The spermine-based or serine-based cationic polymers discussed above were first conjugated with fatty acid(s), cholesterol, phospholipid(s) as described in Example 4. Then aqueous solutions of the hydrophobic anchor-bearing cationic polymer and that of DNA or RNA were added in an organic solvent continuous phase to form a water-in-oil emulsion under vigorous stirring. Then a phospholipids solution (dissolved in chloroform) was added into the continuous phase, followed by a drying process. Finally, the powders obtained were hydrated under sonication to form lipopolyplex (Refer to FIG. 15). Formation of lipopolyplex was confirmed by Zeta potential measurements which indicated that Zeta potential dropped from 30 mV to 15 mV by treatment with lipids.

### Example 14

### Efficiency in Silencing EGPF Gene

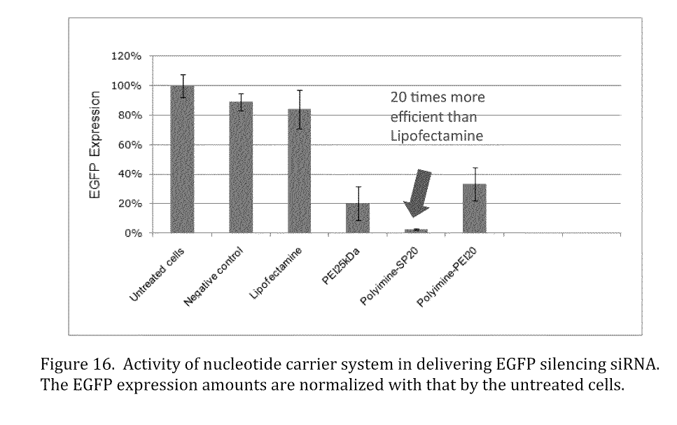

Gene expressing and silencing are not only opposite processes, but also different in the destination to where nucleotide is required to deliver. For the former purpose, a DNA plasmid should be delivered to the nuclei of target cells, while for the latter, a knockout siRNA should be delivered to the cytoplasm of the target cells. To further examine the capability of spermine (or spermidine) based cationic polymer in siRNA delivery, a siRNA of EGFP gene knockout sequence was condensed with various carrier materials and incubated with COS-7 cells. As shown in FIG. 16, the spermine-based cationic polymer (Polyimine-SP20, imine linkage and 20 KDa in molecular weight) was 20 times more efficient than Lipofectamine and 5 times more than PEI, two frequently used transfection agents. This result may be attributed to relatively rapid degradation of the imine linkage of the spermine-based polymer, facilitating the release of siRNA in the cytoplasm.

### REFERENCES

[1] Mulligan, R. C.; The basic science of gene therapy, Science 1993, 260, 926-932.
[2] Pack, D. W.; Hoffman, A. S.; Pun, S.; Stayton, P.; Nature Reviews Drug Discovery 2005, 4, 581-593.
[3] Luo, D.; Saltzman, W. M. Synthetic DNA delivery system. Nature Biotechnology 2000, 18, 33-37.
[4] Xu, Y.; Azoka, Jr. F. C.; Mechanism of DNA release from cationic liposome/DNA complex used in cell transfection. Biochemistry 1996, 35, 5616-5623.
[5] Abdelhady, H. G.; et al.; Direct real-time molecular scale visualization of the degradation of condensed DNA complexes exposed to DNase I. Nucleic Acids Res.; 2003, 31, 4001-4005.
[6] Yokoyama, M.; Gene delivery using temperature responsive polymeric carriers. Drug Discovery Today; 2002, 7, 426-432.
[7] Schaffer, D. V.; Fidelman, N. A.; Dan, N.; Lauffenberger, D. A.; Vector unpacking as a potential barrier for receptor-mediated polyplex gene delivery. Biotechnol. Bioeng.; 2000, 67, 598-606.
[8] Hinrichs, W. L. J.; et al. Thermosensitive polymers as carries for DNA delivery. J. Control. Release 1999, 60, 249-259.
[9] Lim, Y. L.; Kim, S.; Suh, H.; Park, J.; Biodegradable, endosome disruptive, and cationic network-type polymer as a highly efficient and nontoxic gene delivery carrier. Bioconjug. Chem. 2002, 13, 952-957.
[10] Lim, Y. L.; et al.; Biodegradable Polyester, Poly[α-(4-Aminobutyl)-L-glycolic acid], as a Non-Toxic Gene Carrier. Pharm. Res.; 2000, 17, 811-816.
[11] Forrest, M. L.; Koerber, J. T.; Pack, D. W.; A degradable polyethylenimine derivative with low toxicity for highly efficient gene delivery. 2003, 14, 934-940.
[12] Gosselin, M. A.; Guo, W.; Lee, R. J.; Efficient gene transfer using reversibly cross-linked low molecular weight polyethylenimine. Bioconjug. Chem.; 2001, 12, 989-994.
[13] Baker. A.; Gotten. M. Polyethylenimine (PEI) is a simple, inexpensive and effective reagent for condensing and linking plasmid DNA to adenovirus for gene delivery. Gene Therapy, 1997, 4 (8), 773-782.
[14] Jin, T.; Pennefather, P.; Lee, P. I.; Lipobeads: A hydrogel anchored lipid vesicle system. FEBS Letters, 397: 70-74 (1996).

What is claimed is:

1. A cationic polymer comprising a plurality of amine monomers linked together by themselves via cleavable bonds, or by way of linkers, wherein each linker forms cleavable bonds with two or three said amine monomers.

2. The cationic polymer of claim 1, wherein the amine monomers are human endogenous amines or their derivatives.

3. The cationic polymer of claim 1, wherein the amine monomers are selected from spermine, spermidine, serine, N,N-dimethylserine, amino acids, alkylated or dialkylated amino acids, histidine and their combinations thereof.

4. The cationic polymer of claim 1, wherein the cleavable bonds can be cleaved to release said amine monomers.

5. The cationic polymer of claim 1, wherein the cleavable bonds are esters, amides, carbamates or imines.

6. The cationic polymer of claim 1, wherein the linkers comprise an amino group with pKa<8.

7. The cationic polymer of claim 6, wherein the amino group with pKa<8 is an imidazole, amino acid or histidine.

8. The cationic polymer of claim 1, wherein the polymer is conjugated with one or more biologically functional groups.

9. The cationic polymer of claim 8, wherein the one or more biologically functional groups are selected from fatty acids, cholesterol succinate, phospholipids, polyethylene glycol (PEG) and cell-targeting moieties.

10. The cationic polymer of claim 1, wherein the cleavable bonds are formed by reaction between said amine monomers with the following moieties in said linkers: carboxylic acid, aldehyde, chloroformate, acyl halide, ester, or a combination thereof.

11. A method of synthesizing the cationic polymer of claim 1, comprising a reaction between said amine monomers and said linkers possessing two or three reactive groups, or a reaction between said amine monomers.

12. The method of claim 11, wherein the reactive groups are chloroformates.

13. The method of claim 11, wherein the reactive groups are aldehydes.

14. The method of claim 11, wherein the reactive groups are carboxylic acid halides.

15. The method of claim 11, wherein the reactive groups are activated esters.

16. The method of claim 11, wherein the amine monomers react with themselves through ring opening polymerization.

17. The method of claim 11, wherein the amine monomers react with themselves through activated acid-hydroxyl condensation.

18. Use of the cationic polymer of claim 1 to encapsulate and deliver a DNA or RNA.

19. Use of the cationic polymer of claim 9 to assemble lipid bilayers around polyplex through hydrophobic interactions.
